(19) United States
(12) Patent Application Publication
(10) Pub. No.: US 2004/0143452 A1
(43) Pub. Date: Jul. 22, 2004

(54) System and method of dynamically assessing foot characteristics

(75) Inventors: David A. Pattillo, Atlanta, GA (US); Benjamin L. Simon, Atlanta, GA (US)

Correspondence Address: Finnegan, Henderson, Farabow,, Garrett & Dunner, L.L.P., 1300 I Street, N.W., Washington, DC, 20005-3315, US

(73) Assignee: Podo Technology, Inc.

(21) Appl. No.: 10/755,536

(22) Filed: Jan. 12, 2004

Related U.S. Application Data
(60) Provisional application No. 60/440,032, filed on Jan. 15, 2003.

(51) Int. Cl. G06F 17/60; A61B 5/103

(52) U.S. Cl. 705/1; 600/592; 600/595

(57) ABSTRACT

A method for dynamically analyzing an individual's foot during a stride of the foot. The method receives a plurality of pressure readings taken from a pressure platform adapted to be stridden upon by the foot, wherein the pressure readings comprise a position value, a pressure value and a time; determines a cushioning requirement based on the plurality of pressure readings; determines a pronation requirement based on the plurality of pressure readings; and suggests a level of cushioning and a degree of pronation based upon the cushioning requirement and the pronation requirement

### RELATED APPLICATIONS

This patent application claims the benefit of priority of U.S. Provisional Application No. 60/440,032, filed Jan. 15, 2003, which is incorporated herein by reference.

### TECHNICAL FIELD

The invention relates to the field of foot analysis, and more specifically, to a system for and method of dynamically analyzing a foot during a stride in order to determine foot characteristics. Once foot characteristics are determined, appropriate footwear may be suggested.

### BACKGROUND

The world is becoming more interested in exercise, and running, in particular, has grown beyond a cult following to a practice of the masses. In order to cater to this growing market segment, running show manufacturers offer a wide variety of shoes with characteristics designed to meet the individual needs of each individual runner. Manufacturers offer shoes with varying degrees of cushioning, low, moderate, and heavy, to meet individual needs. In addition, there are shoes with varying degrees of pronation, or roll-in, control. Those individuals who heavily overpronate can purchase shoes with a large post inside the arch to help to control the pronation and stabilize the foot in an upright position. Posts are offered in ever decreasing sizes to fit the pronation needs of the purchaser.

The variety of shoes offered by manufacturers serves no end if the individual runner purchases the wrong shoe. An individual walking into an athletic shoe retail store faces a bewildering array of choices: heavily cushioned shoes with a large post, lightly cushioned shoes with a large post, lightly cushioned shoes with no post, etc. A dizzying number of combinations of pronation control and cushioning leaves an individual seeking guidance as to what shoe purchase is appropriate for her feet.

But, the knowledge base of the sales staff in most retail athletic shoe stores in generally not up to the task at hand, and here is the weak link in the chain. At best, a sales person may ask a customer about whether he overpronates or underpronates, or may ask how much cushioning the customer likes. But, at best, the sales person only learns the customer's best guess, or subjective opinion, as to what type of stride she has and what type of show she needs. At worse, and as is most common, the sales person does not even know the correct questions to ask, and a customer is left purchasing a shoe based on looks, price, or the opinion of a friend who may have a very different stride.

In order to overcome the subjective nature of fitting an athlete for a shoe, those skilled in the art have attempted more objective, computerized, approaches to fitting an athletic shoe. Generally, the subject person stands on a platform that would then analyze his foot. This static analysis might measure the pressure at various points on the bottom of the foot to develop a pressure map of the standing foot, or, in more sophisticated systems, the static analysis might also include three dimensional imagery of the static foot. Based on these static analyses, recommendations or a fitting might be made.

However, these prior art systems performed analysis based solely on static data. None of the prior art systems analyzed a foot dynamically, i.e. during a person's stride. Data taken statically fails to truly analyze the dynamics of a foot during running or exercising, which is the time for which the analysis is truly valuable and needed. A prior art system by the present inventors provides for display of dynamic data from a foot stride, but fails to perform any automated analysis thus relying on a trained individual to interpret the data. Such a system fails in the retail environment where sales personnel are rarely properly trained.

The present invention is directed to overcoming the one or more problems or disadvantages associated with the prior art.

### SUMMARY

In accordance with an aspect of the present invention, a method for dynamically analyzing an individual's foot during a stride of the foot is provided. The method receives a plurality of pressure readings taken from a pressure platform adapted to be stridden upon by the foot, wherein the pressure readings may comprise a position value, a pressure value and a time. The method further determines a cushioning requirement based on the plurality of pressure readings; and determines a pronation requirement based on the plurality of pressure readings.

In accordance with another aspect of the present invention, a machine-readable storage medium having stored thereon machine executable instructions is provided. The execution of the instructions is adapted to implement a method for dynamically analyzing an individual's foot during a stride of the foot. The method comprises receiving a plurality of pressure readings taken from a pressure platform adapted to be stridden upon by the foot, wherein the pressure readings comprise a position value, a pressure value and a time; determining a cushioning requirement based on the plurality of pressure readings; and determining a pronation requirement based on the plurality of pressure readings.

In accordance with yet another aspect of the present invention, a system for dynamically analyzing an individual's foot during a stride of the foot is provided. A pressure platform is adapted to be stridden upon by the foot. A microprocessor is coupled to the pressure platform and coupled a memory. The microprocessor is operable to: receive a plurality of pressure readings taken from the pressure platform, wherein the pressure readings comprise a position value, a pressure value and a time; determine a cushioning requirement based on the plurality of pressure readings; and determine a pronation requirement based on the plurality of pressure readings.

The foregoing summarizes only a few aspects of the invention and is not intended to be reflective of the full scope of the invention as claimed. Additional features and advantages of the invention are set forth in the following description, may be apparent from the description, or may be learned by practicing the invention. Moreover, both the foregoing summary and the following detailed description are exemplary and explanatory and are intended to provide further explanation of the invention as claimed.

### BRIEF DESCRIPTION OF THE DRAWINGS

The accompanying drawings, which are incorporated in and constitute a part of this specification, illustrate one embodiment of the invention and together with the description, serve to explain the principles of the invention.

FIG. 1 illustrates a system environment in which the features and principles of the present invention may be implemented.

FIG. 2 illustrates a computer system consistent with the principals of the present invention.

FIG. 3 is a flow chart illustrating the process of dynamically analyzing a foot consistent with the principals of the present invention.

FIG. 4 is a flow chart illustrating the process of determining cushioning requirements consistent with the principals of the present invention.

FIG. 5 is a flow chart illustrating the process of determining cushioning requirements based on forefoot pressure consistent with the principals of the present invention.

FIG. 6 is a pressure graph illustrating pressure readings and the division of the foot into forefoot, arch area and heel consistent with the principals of the present invention.

FIG. 7 is a flow chart illustrating the process of determining cushioning requirements based on the speed through the forefoot consistent with the principals of the present invention.

FIG. 8 is a flow chart illustrating the process of determining pronation requirements consistent with the principals of the present invention.

FIG. 9 is a flow chart illustrating the process of analyzing the speed of the foot through the arch to determine pronation requirements consistent with the principals of the present invention.

FIG. 10 is a flow chart illustrating the process of analyzing the pressure on the inside of the forefoot to determine pronation requirements consistent with the principals of the present invention.

FIG. 11 is a flow chart illustrating the process of analyzing the pressure on the foot in the arch to determine pronation requirements consistent with the principals of the present invention.

FIG. 12 is a diagram of pressure in the arch area of the foot and midline of the arch area consistent with the principals of the present invention.

FIG. 13 is a flow chart illustrating the process of analyzing the gait to determine pronation requirements consistent with the principals of the present invention.

FIG. 14 is a diagram of the gait line consistent with the principals of the present invention.

### DETAILED DESCRIPTION

Reference will now be made in detail to embodiments consistent with the present invention, examples of which are illustrated in the accompanying drawings. Wherever possible, the same reference numbers will be used throughout the drawings to refer to the same or like parts.

In general, the present invention provides a method of dynamically analyzing a foot during a stride in order to provide analysis of the cushioning requirements or pronation requirements of an individual's foot. This technique may also be referred to as dynamic gait analysis. Using this information, the method may determine appropriate shoes based on the individual's cushioning requirements or pronation requirements and suggest shoes to the individual.

The system may receive pressure readings from a pressure platform which provides a plurality of pressure readings, where each pressure. reading may comprise a position value, a pressure value, and a time value. These readings are taken as an individual strides upon and across the platform.

Cushioning requirements may range, for example, from low to moderate to high and may be calculated by measuring the pressure through the forefoot and/or the speed of the foot through the forefoot. In general, the more pressure placed upon the forefoot, the greater the degree of cushioning required. In addition, the slower the speed of the foot through the forefoot, the greater the cushioning required.

Pronation requirements may range from severe overpronation to moderate overpronation to neutral pronation to underpronation. In general, the present invention categorizes pronation values, for example, from 5 to 0, where 5 is severe overpronation and 0 is no overpronation. Pronation requirements may be calculated based on one or more of the following determinations: speed of the foot through the arch, pressure on the inside of the forefoot; pressure of the foot in the arch; and/or gait. The method may use one or more of the above determinations, either alone or in combination, to determine pronation requirements.

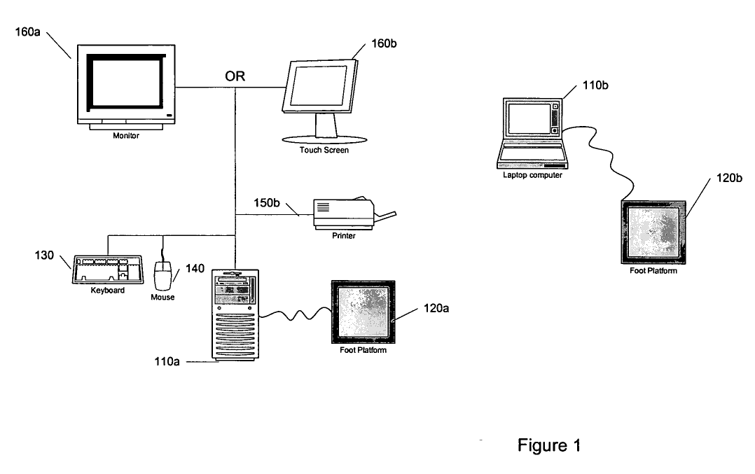

FIG. 1 illustrates a system environment in which the features and principles of the present invention may be implemented. The system may comprise a computer system 110 coupled to a pressure platform 120. The pressure platform may be coupled via Universal Serial Bus, USB2, Firewire, PCI, or serial connection, for example. A pressure platform utilized consistent with the principals of the invention is the Medicapteurs PEL 38 Platform. This platform can acquire pressure data from a 32×32 sensor pad of sensors of 1 cm.×1 cm. at over 100 images per second, thus making it particularly appropriate to the dynamic nature of the present invention. Further advanced Medicapteurs platforms suitable for use with the invention feature a 48×48 sensor pad of sensors of 0.8 cm×0.8 cm. Other sensor pads with greater resolution, for example, may also be used.

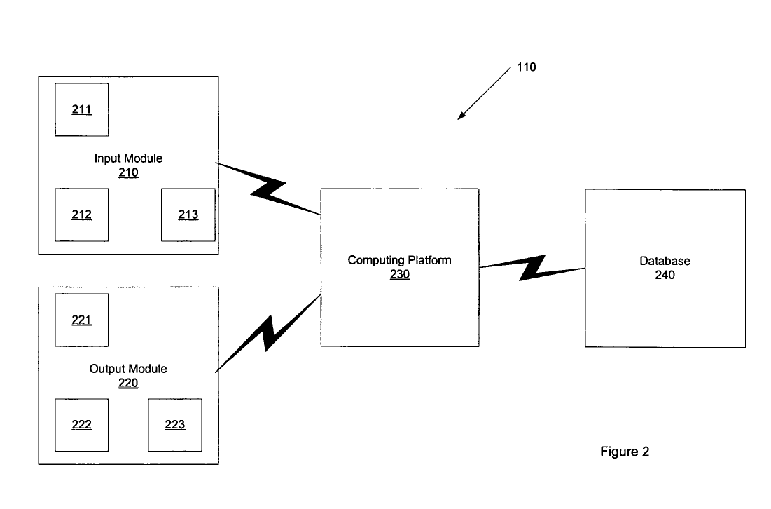

FIG. 2 illustrates a computer system 110 consistent with the principals of the present invention. In this embodiment, computer systems, such as systems 110a and 110b, operate the method of the present invention. Computer system 110a may be coupled to a keyboard 130 and mouse 140, as well as display 160. As illustrated in the block diagram of FIG. 2, a system environment consistent with an embodiment of the present invention may include an input module 210, an output module 220, a computing platform 230, and a database 240. Computing platform 230 is adapted to include the necessary functionality and computing capabilities to implement the dynamic foot analysis methodology through input module 210 which is coupled to pressure platform 120 (FIG. 1) and access, read and write to database 240. The results of analyzing the pressure readings are provided as output from computing platform 230 to output module 220 for printed display, viewing, or further communication to other system devices. Such output may include, for example, one or more requirements printouts or shoe recommendations. Output from computing platform 230 can also be provided to database 240, which may be utilized as a persistent storage device for storing, for example, individual pressure readings taken during a stride.

In the embodiment of FIG. 2, computing platform 230 may comprise a PC or PDA for performing various functions and operations of the invention. Computing platform 230 may be implemented, for example, by a general purpose computer selectively activated or reconfigured by a computer program stored in the computer, or may be a specially constructed computing platform for carrying-out the features and operations of the present invention. Computing platform 230 may also be implemented or provided with a wide variety of components or subsystems including, for example, one or more of the following: one or more central processing units, a co-processor, memory, registers, and other data processing devices and subsystems. Computing platform 230 also communicates or transfers dynamic analysis input and output to and from input module 210 and output module 220 through the use of direct connections or communication links, as illustrated in FIG. 2.

Alternatively, communication between computing platform 230 and modules 210, 220 can be achieved through the use of a network architecture (not shown). In the alternative embodiment (not shown), the network architecture may comprise, alone or in any suitable combination, a telephone-based network (such as a PBX or POTS), a local area network (LAN), a wide area network (WAN), a dedicated intranet, and/or the Internet. Further, it may comprise any suitable combination of wired and/or wireless components and systems. By using dedicated communication links or shared network architecture, computing platform 230 may be located in the same location or at a geographically distant location from input module 210 and/or output module 220.

Input module 210 of the system environment shown in FIG. 2 may be implemented with a wide variety of devices to receive and/or provide the data as input to computing platform 230. As illustrated in FIG. 2, input module 210 includes an input device 211, a storage device 212, and/or a network 213. Input device 211 may include a keyboard, a mouse, a disk drive, video camera, magnetic card reader, or any other suitable input device for providing customer information to computing platform 230. Memory device may be implemented with various forms of memory or storage devices, such as read-only memory (ROM) devices and random access memory (RAM) devices. Storage device 212 may include a memory tape or disk drive for reading and providing information on a storage tape or disk as input to computing platform 220. Input module 210 may also include network interface 213, as illustrated in FIG. 2, to receive data over a network (such as a LAN, WAN, intranet or the Internet) and to provide the same as input to computing platform 230. For example, network interface 213 may be connected to a public or private database over a network for the purpose of receiving information about the customers from computing platform 230.

As illustrated in FIG. 2, output module 220 includes a display adapter 221, a printer device adapter 222, and/or a network interface 223 for receiving the results provided as output from computing module 220. The output from computing platform 230 may be displayed or viewed through display adapter 221 (such as a CRT or LCD) and printer device adapter 222. If needed, network interface 223 may also be provided to facilitate the communication of the results from computer platform 230 over a network (such as a LAN, WAN, intranet or the Internet) to remote or distant locations for further analysis or viewing.

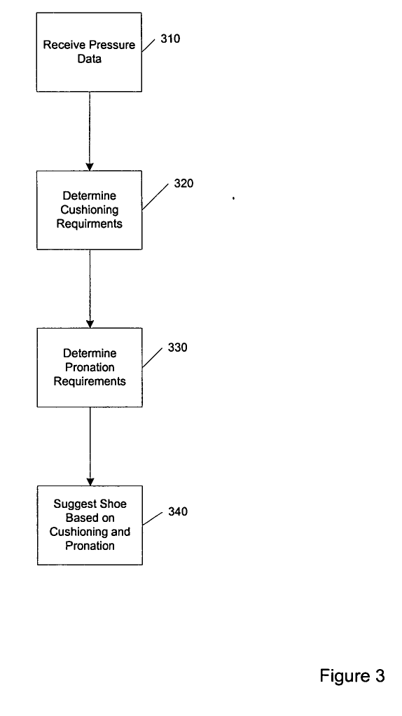

FIG. 3 is a flow chart illustrating a process of dynamically analyzing a foot consistent with the principals of the present invention. At stage 310, the method receives pressure readings from the pressure platform. The pressure readings may comprise a position value, a pressure value, and a time value. The position value may comprise, for example, an x-coordinate and a y-coordinate or may be in polar notation. In this way, dynamic reading are receiving based on an individual striding across the platform. Consistent with the principals of the present invention, the pressure readings values may be normalized to a value of between 0 and 100.

At stage 320, the method calculates the cushioning requirements based on the pressure readings. At stage 330, the method calculates the pronation requirements based on the pressure readings. At stage 340, the method provides the individual with suggested level of cushioning and pronation based on the cushioning and pronation requirements. Methods consistent with the principles of the present invention for performing stages 320 and 330 are further illustrated below. Other methods may also be appropriate for calculating these values.

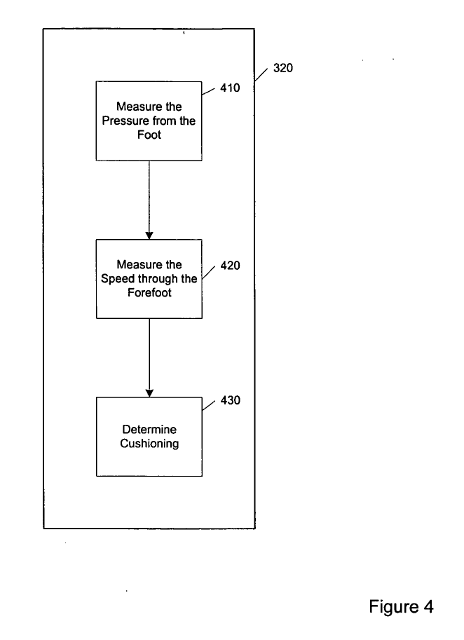

FIG. 4 is a flow chart illustrating the process of determining cushioning requirements consistent with the principals of the present invention. Measuring the pressure of the foot at the forefoot and/or measuring the speed of the foot through the forefoot may be used to calculate cushioning. The relative speed of portions of the foot can change as a person strides. For example, a striding foot may remain on the heel for a long period of time, then rapidly move through the forefoot. The speed of the foot at various points is the rate of change of the position of the foot from a first point on the bottom of the foot to a second point on the bottom of the foot. At stage 410, the method measures the pressure in the forefoot to preliminarily calculate the cushioning requirements. The larger the pressure on the forefoot, the greater the degree of cushioning required. At stage 420, the method calculates the speed of the foot through the forefoot to modify cushioning requirements. In general, the slower the speed through the forefoot, the greater the cushioning required.

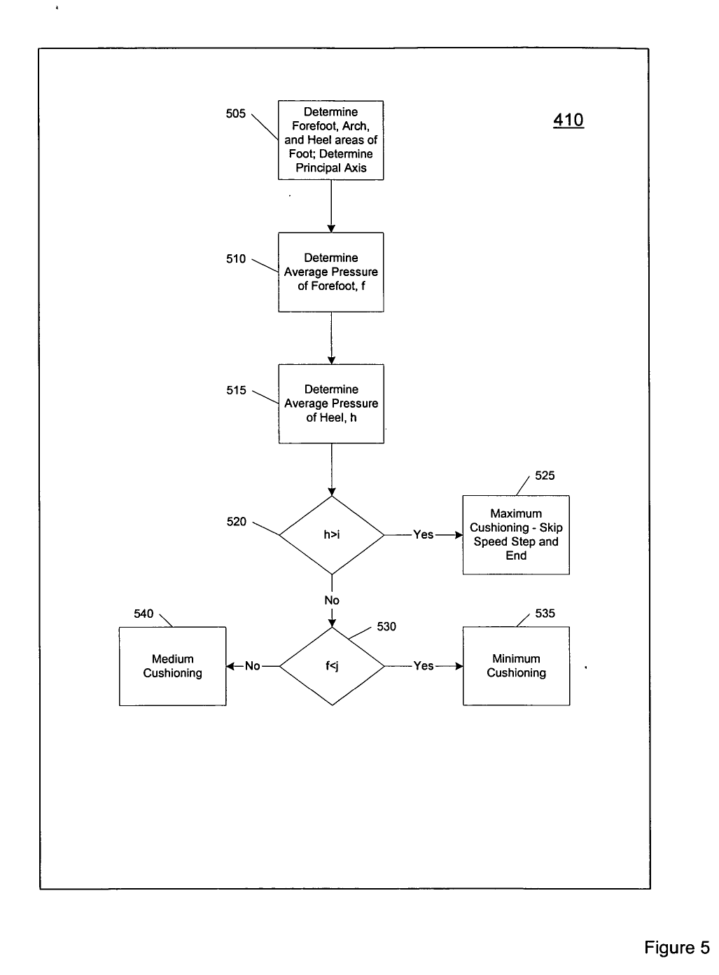

FIG. 5 is a flow chart illustrating the process of determining cushioning requirements based on forefoot pressure consistent with the principals of the present invention. At stage 505, the method examines a pressure map 600 and determines the forefoot, arch, and heel areas of the foot embodied within pressure map 600. In addition, the principal axis of the foot is determined. These values may be used in this and other sub-methods of the present invention.

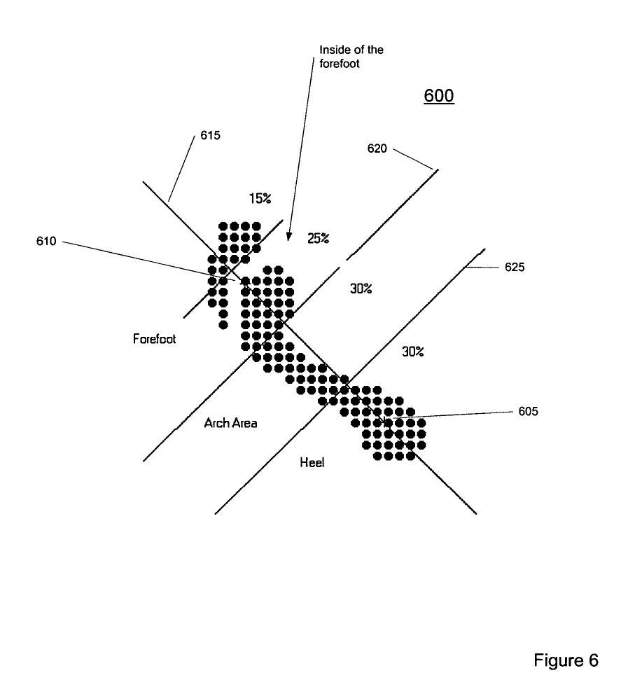

A slight digression into FIG. 6 will be useful in explaining the principles of the present invention. FIG. 6 is a graph illustrating a pressure map 600 and the division of the plantar portion of the foot into a forefoot area, an arch area and heel area consistent with the principals of the present invention. The forefoot area is determined to be the distal plantar area of the foot, between the most distal portion of the plantar portion of the foot and generally, 30-50% lateral of the most distal portion of the foot, or more particularly 40% from the distal portion of the foot. At this measurement, the forefoot line 620 is drawn. The arch area of the foot is between the forefoot line and generally, 60-80% from the most distal portion of the plantar portion of the foot, or more particularly 70% from the most distal portion of the foot. At this measurement the arch line 625 is drawn. The remaining planar portion of the foot is the heel area of the foot. Forefoot midpoint 610 is illustrated in the center of the forefoot area about 20% from the most distal portion of the plantar portion of the foot. Heel midpoint 605 is illustrated in the center of the heel portion about 15% from the most lateral point of the planar part of the foot. Principal axis 615 is drawn between the forefoot midpoint and the heel midpoint. The inside of the foot is the portion of the foot inside of the principal axis 615.

Returning to FIG. 5, at stage 510, the average forefoot pressure, f, is calculated from those pressure readings falling within the forefoot area. At stage 515, the average heel pressure, h, is calculated from those pressure readings falling within the heel area. At stage 520, the average heel pressure is compared to heel trigger point, i. Heel trigger point i is established such that at a point greater than i high heel cushioning would be beneficial. If heel pressure, h, is greater than heel trigger pressure, i, then high cushioning is required and it is unnecessary to perform stage 410 (stage 525).

If not, at stage 530 forefoot pressure, f, is compared to forefoot trigger point, j. Forefoot trigger point j is established such that at a point greater than j moderate cushioning would be beneficial. If forefoot pressure, f, is less than forefoot trigger point, j, then low cushioning is required (stage 535). Else, medium cushioning is required (stage 540).

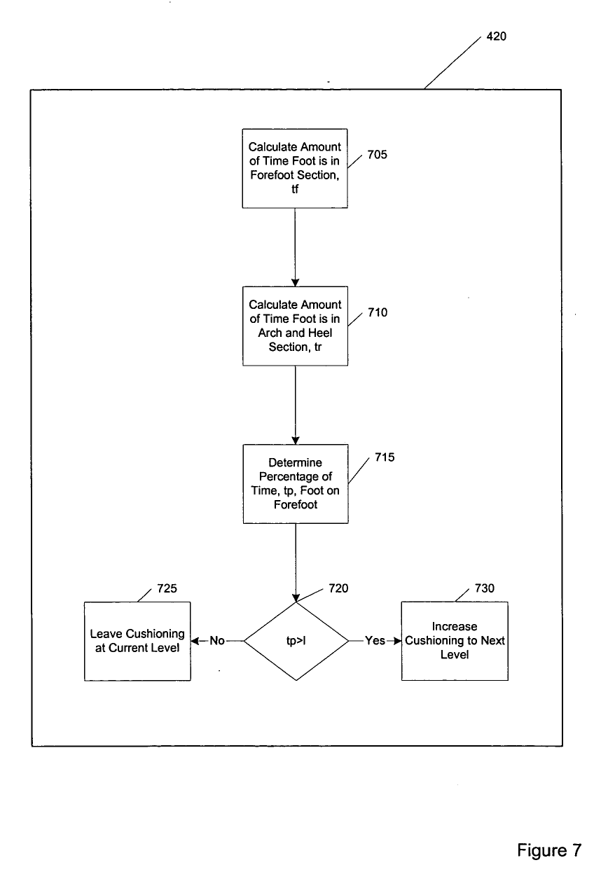

FIG. 7 is a flow chart illustrating the process of determining cushioning requirements based on the speed through the forefoot according to the principals of the present invention. At stage 705, the method calculates the amount of time, tf, that the foot is in the forefoot section during a stride. The method uses the time values within the pressure readings to determine this factor. Those skilled in the art will appreciate that other methods may be used. At stage 720, the method calculates the amount of time, tr, that the foot is in the arch and heel sections of the foot during a stride.

At stage 715, the method determines the percentage of time, tp, that the foot is in the forefoot compared to the remainder of the foot. At stage 720, the percentage of time, tp, is compared to a trigger value, I. Trigger value I is established such that at a percentage greater than I increased cushioning would be beneficial. If the percentage of time, tp, is greater than trigger point, I, then, at stage 730, the previously calculated cushioning requirement is increased one level. For example, if low cushioning was previously calculated in method 410, then the cushioning level is increased to moderate cushioning. At stage 725, if the trigger point, I, is not exceeded, then cushioning remains where previously calculated by method 410.

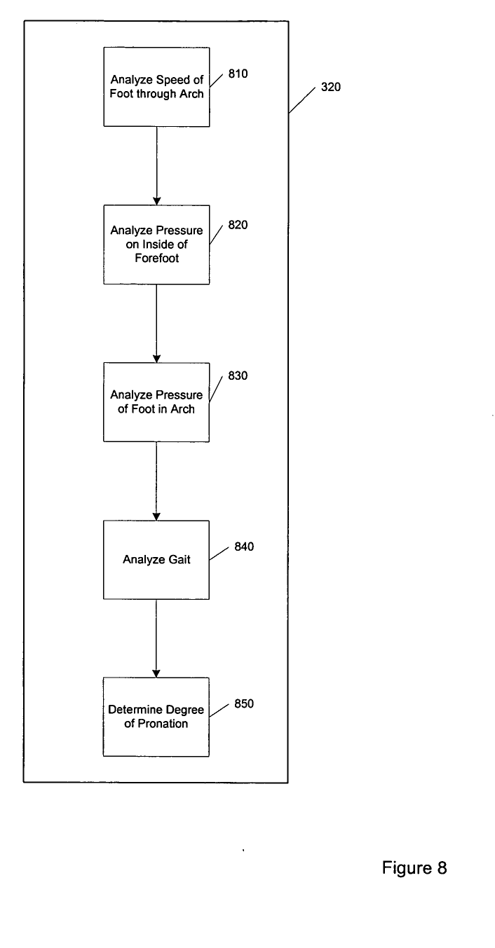

FIG. 8 is a flow chart illustrating the process of determining pronation requirements according to the principals of the present invention. Pronation requirements may be calculated by using one or more of the following sub-methods, either alone or in combination. Stage 810 analyzes the speed of the foot through the arch. Stage 820 analyzes the pressure on the inside of the forefoot. Stage 830 analyzes the pressure of the foot in the arch. And, stage 840 analyzes the gait of the individual. Each sub-method yields a value of between 5 and 0, with 5 indicating severe overpronation and 0 indicating no overpronation. The output of the sub-methods, 810-840, may be combined in sub-method 850 through a weighted average or other method.

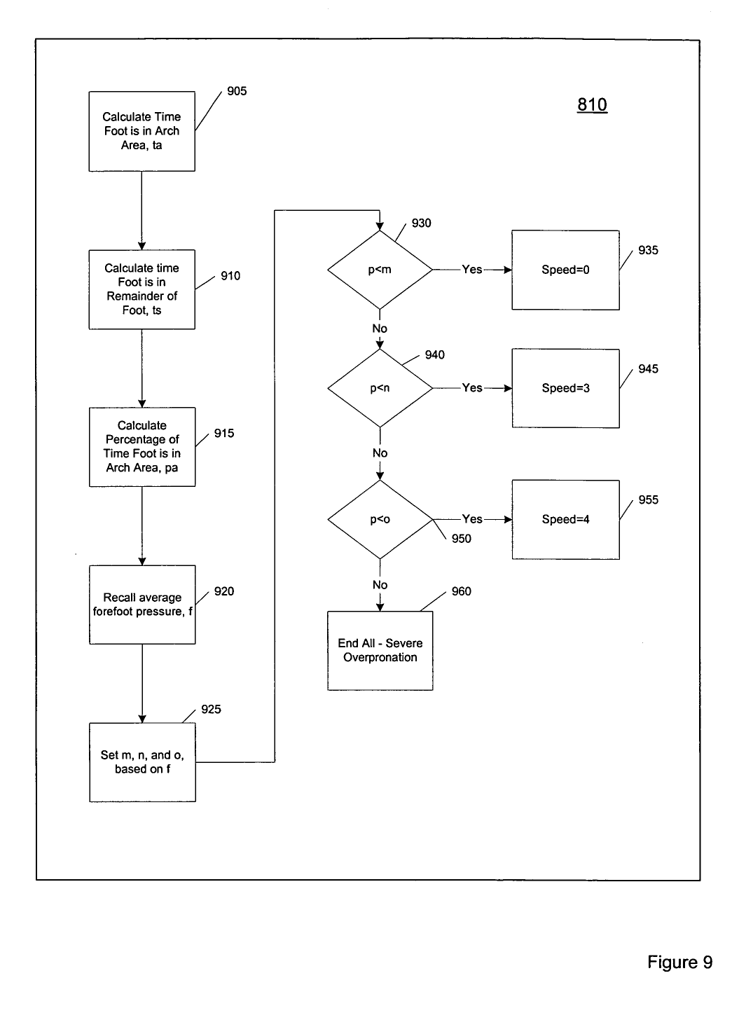

FIG. 9 is a flow chart illustrating the process of analyzing the speed of the foot through the arch to determine pronation requirements consistent with the principals of the present invention. At stage 905, the time the foot is in the arch area, ta, is calculated. At stage 910, the time the foot is in the heel and forefoot, ts, is calculated. At stage 915, the percentage of time the foot is in the arch area, pa, is calculated. At stage 920, the average forefoot pressure, f, is recalled from stage 510. Trigger points m, n, and o are selected based on the forefoot pressure, f, where m<n<o.

At stage 930, if pa<m then the output of this method is set to a pronation value of 0, speed=0 (stage 935). At stage 940, if pa<n then the output of this method is set to a pronation value of 3, speed=3 (stage 945). At stage 950, if pa<o then the output of this method is set to a pronation value of 4, speed=4 (stage 955). If not, at stage 960 severe overpronation is detected and the pronation value is set at 5 with none of the other sub-methods, 820-840, required.

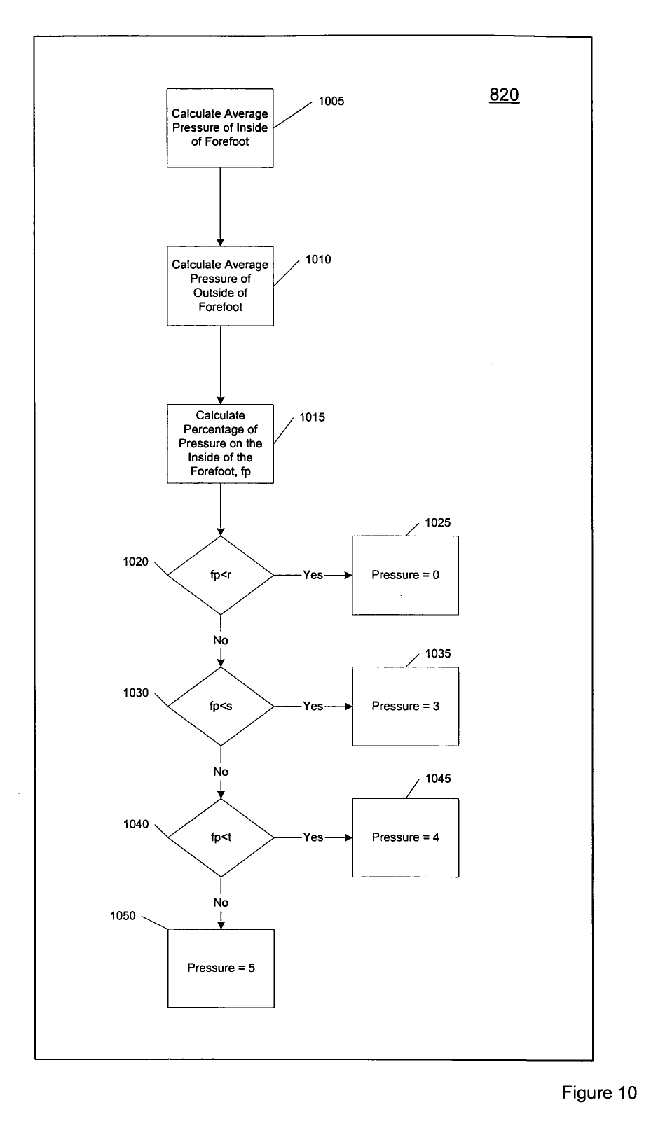

FIG. 10 is a flow chart illustrating the process of analyzing the pressure on the inside of the forefoot to determine pronation requirements according to the principals of the present invention. At stage 1005, the average pressure of the inside of the forefoot is calculated. The inside of the forefoot is that portion of the forefoot on the inside of the principal axis. At stage 1010, the average pressure of the outside of the forefoot is calculated. The outside of the forefoot is that portion of the forefoot on the outside of the principle axis. At stage 1015, the percentage of pressure on the inside of the forefoot, fp, compared with the total pressure on the forefoot is calculated.

At stage 1020, if forefoot percentage pressure, fp, is less than trigger point r, then at stage 1025 pronation results are set to 0, Pressure=0. At stage 1030, if forefoot percentage pressure, fp, is less than trigger point s, then at stage 1035 pronation results are set to 3, Pressure=3. At stage 1040, if forefoot percentage pressure, fp, is less than trigger point t, then at stage 1045 pronation results are set to 4, Pressure=4. Otherwise, pronation results of this sub-method are set to 5, Pressure=5 (stage 1050).

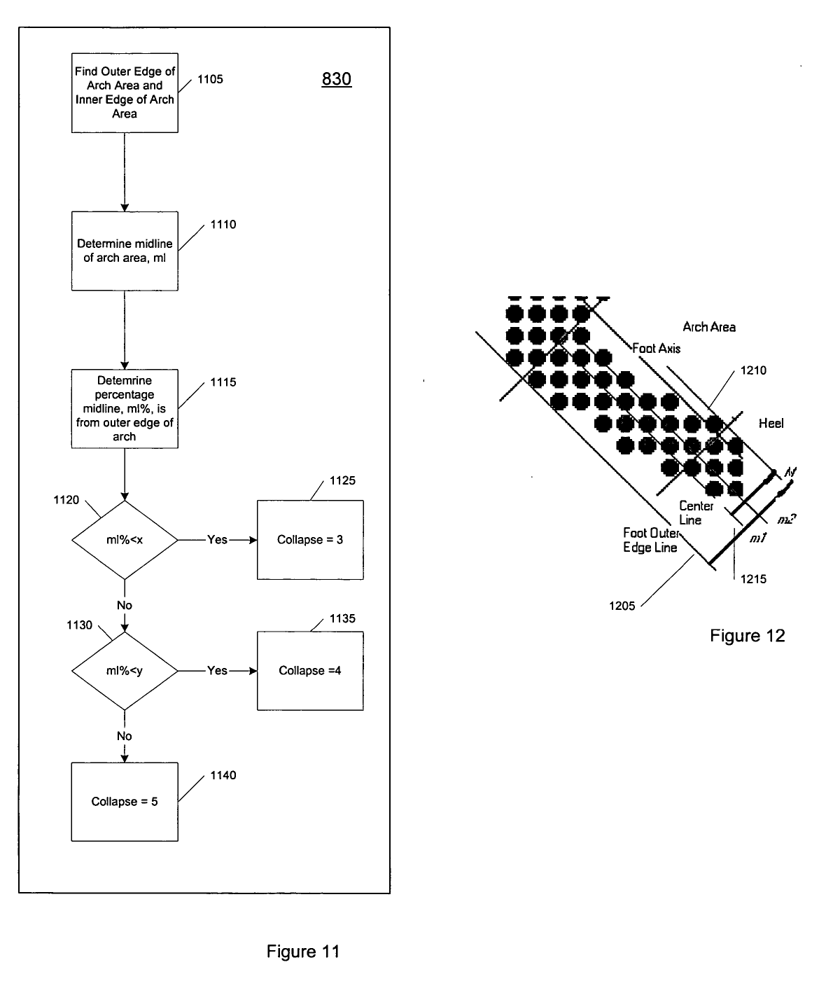

FIG. 11 is a flow chart illustrating the process of analyzing the pressure on the foot in the arch to determine pronation requirements according to the principals of the present invention. At stage 1105, the outer edge of the arch area 1205 and the inner edge of the arch area 1210 (FIG. 12) is found. At stage 1110, the midline 1215 (FIG. 12) is found between the outer edge and the inner edge. At stage 1115, the midline percentage, ml %, is calculated based on the percentage distance that the midline is from the outer edge of the arch.

At stage 1120, if midline percentage, ml %, is less than trigger point x, then at stage 1125 pronation results are set to 3, Collapse=3. At stage 1130, if midline percentage, ml %, is less than trigger point y, then at stage 1135 pronation results are set to 4, Collapse=4. Otherwise, pronation results of this sub-method are set to 5, Collapse=5 (stage 1140).

FIG. 12 is a diagram of pressure in the arch area of the foot and midline of the arch area according to the principals of the present invention. The outer edge of the arch is illustrated as 1205. The inner edge of the arch is illustrated as 1210. The midline 1215 is placed between these two lines.

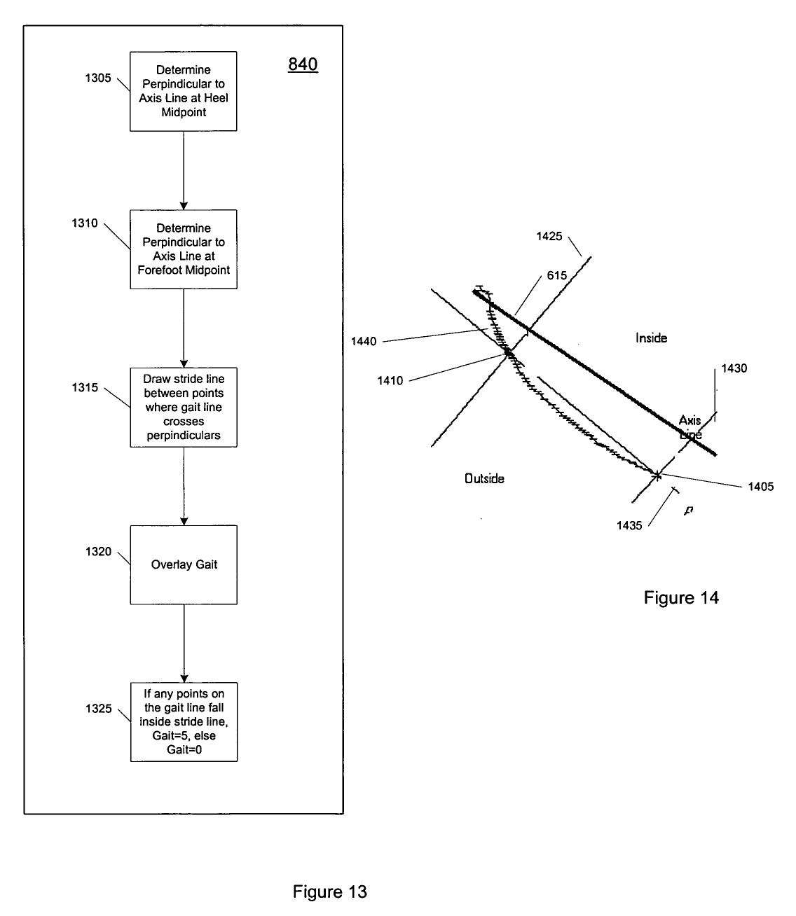

FIG. 13 is a flow chart illustrating the process of analyzing the gait to determine pronation requirements according to the principals of the present invention. In general, stage 840 analyzes the gait line to detect any rolling in of the foot that may be missed by other stages, 810-830. To illustrate FIG. 13, FIG. 14 is a diagram of the gait line consistent with the principals of the present invention. The gait line 1440 is derived based on the pressure readings and is an indication of the center of pressure of the individual through the stride, where the center of pressure is the average point of pressure exerted by the foot on the platform at time, tᵢ. The principal axis is illustrated as 615. A forefoot perpendicular line 1425 extends perpendicular to the principal axis 615 at forefoot midpoint 610. A heel perpendicular line 1430 extends perpendicular to the principal axis 615 at heel midpoint 605. A gait perpendicular 1435 extends between the crossing points of the gait line 1440 and the perpendiculars 1425 and 1430.

Returning to FIG. 13, at stage 1305, the heel perpendicular is found as illustrated above. At stage 1310, the forefoot perpendicular is found as illustrated above. At stage 1315, the gait perpendicular 1435 is calculated as shown above. At stage 1320, the gait line is overlaid and compared to the gait perpendicular. At stage 1325, if any points on the gait line fall between the gait perpendicular 1435 and the principal axis 615, then pronation value is a 5, Stride=5, else the pronation value of this sub-method is a zero.

Once these four pronation values are found, in stages 810-840, at stage 850 they may be combined through a weighting process for example and used to determine a pronation requirement.

Following the calculation of the cushioning requirement and the pronation requirement, the system may utilize a database of shoes to provide a sample listing of proper shoes. The database may reside in database 240 which may be updated in a variety of ways known to those skilled in the art. The database may categorize shoes by cushioning requirement and pronation requirement. For example, a shoe ABC may be associated with a cushioning requirement of low and a pronation requirement ranging from 0 to 2.

The foregoing description of embodiments of the invention has been presented for purposes of illustration and description. It is not exhaustive and does not limit the invention to the precise form disclosed. Modifications and variations are possible in light of the above teachings or may be acquired from practicing of the invention. For example, the described implementation includes a particular computer configuration but the present invention may be implemented in a variety of electronic systems using software, hardware, or a combination of hardware and software to provide the processing functions.

Those skilled in the art will appreciate that all or part of systems and methods consistent with the present invention may be stored on or read from other computer-readable media, such as: secondary storage devices, like hard disks, floppy disks, and CD-ROM; a carrier wave received from the Internet; or other forms of computer-readable memory, such as read-only memory (ROM) or random-access memory (RAM).

Furthermore, one skilled in the art will also realize that the processes illustrated in this description may be implemented in a variety of ways and include multiple other modules, programs, applications, scripts, processes, threads, or code sections that all functionally interrelate with each other to accomplish the individual tasks described above for each module, script, and daemon. For example, it is contemplated that these programs modules may be implemented using commercially available software tools, using custom object-oriented code written in the C++ programming language, using applets written in the Java programming language, or may be implemented as with discrete electrical components or as one or more hardwired application specific integrated circuits (ASIC) custom designed just for this purpose.

It will be readily apparent to those skilled in this art that various changes and modifications of an obvious nature may be made, and all such changes and modifications are considered to fall within the scope of the appended claims. Other embodiments of the invention will be apparent to those skilled in the art from consideration of the specification and practice of the invention disclosed herein. It is intended that the specification and examples be considered as exemplary only, with a true scope and spirit of the invention being indicated by the following claims and their equivalents.

What is claimed is:

1. A method for dynamically analyzing an individual's foot during a stride of the foot, the method comprising:
receiving a plurality of pressure readings wherein the pressure readings comprise a position value, a pressure value and a time;
determining a cushioning requirement based on the plurality of pressure readings; and
determining a pronation requirement based on the plurality of pressure readings.

2. The method of claim 1, further comprising determining a level of cushioning based upon the cushioning requirement.

3. The method of claim 1, further comprising determining a degree of pronation based upon the pronation requirement.

4. The method of claim 1, further comprising determining a level of cushioning and a degree of pronation based upon the cushioning requirement and the pronation requirement.

5. The method of claim 4, further comprising determining a recommended shoe based on the level of cushioning and the degree of pronation.

6. The method of claim 4, wherein determining the cushioning requirement further comprises:
determining an average pressure of a forefoot of the foot;
determining an average pressure of a heel of the foot;
if the average pressure of the heel of the foot is greater than a high threshold value, setting the cushioning requirement as high; and
if the average pressure of the forefoot is less than a low threshold value, setting the cushioning requirement as low, and, if not, setting the cushioning requirement as medium.

7. The method of claim 6, wherein determining the cushioning requirement further comprises determining a speed of the forefoot and adjusting the cushioning requirement based on the measured speed of the forefoot.

8. The method of claim 7, wherein adjusting the cushioning requirement further comprises increasing the cushioning requirement if the speed of the forefoot is greater than a forefoot speed threshold value.

9. The method of claim 4, wherein determining the pronation requirement further comprises:
analyzing a speed of the foot through the arch;
analyzing a pressure on the inside of the foot;
analyzing a pressure of the foot in the arch;
analyzing the gait of the foot; and
calculating the pronation requirement based on the analyzing steps.

10. A machine-readable storage medium having stored thereon machine executable instructions, the execution of the instructions is adapted to implement a method for dynamically analyzing an individual's foot during a stride of the foot, the method comprising:
receiving a plurality of pressure readings wherein the pressure readings comprise a position value, a pressure value and a time;
determining a cushioning requirement based on the plurality of pressure readings; and
determining a pronation requirement based on the plurality of pressure readings.

11. The medium of claim 10, further comprising instructions for determining a level of cushioning based upon the cushioning requirement.

12. The medium of claim 10, further comprising instructions for determining a degree of pronation based upon the pronation requirement.

13. The medium of claim 10, further comprising instructions for determining a level of cushioning and a degree of pronation based upon the cushioning requirement and the pronation requirement.

14. The medium of claim 13, further comprising instructions for determining a recommended shoe based on the level of cushioning and the degree of pronation.

15. The medium of claim 13, further comprising instructions for:
determining an average pressure of the forefoot of the foot;
determining an average pressure of the heel of the foot;
if the average pressure of the heel of the foot is greater than a high threshold value, setting the cushioning requirement as high; and
if the average pressure of the forefoot is less than a low threshold value, setting the cushioning requirement as low, and, if not, setting the cushioning requirement as medium.

16. The medium of claim 15, further comprising instructions for determining a speed of the forefoot and adjusting the cushioning requirement based on the measured speed of the forefoot.

17. The medium of claim 16, further comprising instructions for increasing the cushioning requirement if the speed of the forefoot is greater than a forefoot speed threshold value.

18. The medium of claim 13, further comprising instructions for:
analyzing a speed of the foot through the arch;
analyzing a pressure on the inside of the foot;
analyzing a pressure of the foot in the arch;
analyzing a gait of the foot; and
calculating the pronation requirement based on the analyzing steps.

19. A system for dynamically analyzing an individual's foot during a stride of the foot, the system comprising a memory and a microprocessor coupled to the memory and programmed to:
receive a plurality of pressure readings wherein the pressure readings comprise a position value, a pressure value and a time;
determine a cushioning requirement based on the plurality of pressure readings; and
determine a pronation requirement based on the plurality of pressure readings.

20. The system of claim 19, wherein the microprocessor is further programmed to determine a level of cushioning based upon the cushioning requirement.

21. The system of claim 19, wherein the microprocessor is further programmed to determine a degree of pronation based upon the pronation requirement.

22. The system of claim 19, wherein the microprocessor is further programmed to determine a level of cushioning and a degree of pronation based upon the cushioning requirement and the pronation requirement.

23. The system of claim 22, wherein the microprocessor is further programmed to determine a recommended shoe based on the level of cushioning and the degree of pronation.

24. The system of claim 22, wherein the microprocessor is further programmed to:
determine an average pressure of the forefoot of the foot;
determine an average pressure of the heel of the foot;
if the average pressure of the heel of the foot is greater than a high threshold value, set the cushioning requirement as high; and
if the average pressure of the forefoot is less than a low threshold value, set the cushioning requirement as low, and, if not, set the cushioning requirement as medium.

25. The system of claim 24, wherein the microprocessor is further programmed to determine the speed of the forefoot and adjust the cushioning requirement based on the measured speed of the forefoot.

26. The system of claim 25, wherein the microprocessor is further programmed to increase the cushioning requirement if the speed of the forefoot is greater than a forefoot speed threshold value.

27. The system of claim 22, wherein the microprocessor is further programmed to:
analyze a speed of the foot through the arch;
analyze a pressure on the inside of the foot;
analyze a pressure of the foot in the arch;
analyze a gait of the foot; and
calculate the pronation requirement based on the analyzing steps.
